(19) United States
(12) Patent Application Publication
(10) Pub. No.: US 2007/0129441 A1
(43) Pub. Date: Jun. 7, 2007

(54) PROTECTION OF CELLS FROM ADVERSE EXTERNAL OR INTRINSIC EFFECTS, CELLULAR DEGENERATION AND DEATH BY N-ACYLETHANOLAMINES

(75) Inventor: Peter Koulen, Benbrook, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Ft. Worth, TX (US)

(21) Appl. No.: 11/621,377

(22) Filed: Jan. 9, 2007

Related U.S. Application Data
(63) Continuation-in-part of application No. 10/840,449, filed on May 6, 2004.
(60) Provisional application No. 60/468,160, filed on May 6, 2003.

(51) Int. Cl. A61K 31/16

(52) U.S. Cl. 514/625

(57) ABSTRACT

The present invention includes compositions and methods for treating diseases of the eye and skin by modulating the amount of intracellular calcium using transdermal or transcorneal delivery of one or more N-acylethanolamines in a carrier.

### CROSS-REFERENCE TO RELATED APPLICATIONS

This application is a continuation-in-part application of U.S. application Ser. No. 10/840,449, filed May 6, 2004, which claims priority to U.S. Provisional Application Ser. No. 60/468,160, filed May 6, 2003, the entire contents of each of which are incorporated herein by reference.

### TECHNICAL FIELD OF THE INVENTION

The present invention relates to new compositions and methods for the treatment of neurodegenerative disorders, and more particularly, to the characterization and therapeutic use of modulators of intracellular calcium channel signaling in cellular physiology.

### BACKGROUND OF THE INVENTION

Without limiting the scope of the invention, its background is described in connection with treatments for ocular degeneration.

The term glaucoma describes a group of eye diseases with a broad spectrum of clinical presentations, etiologies and treatment modalities. Generally, glaucoma causes pathological changes in the optic nerve, visible on the optic disk leading to visual field loss and blindness if untreated.

In glaucomas associated with an elevation in eye pressure (intraocular hypertension) the source of resistance to fluid outflow is generally in the trabecular meshwork. Trabecular meshwork tissue is found between the aqueous humor and the Schlemm's canal. Aqueous humor is the transparent liquid that fills the region between the cornea, at the front of the eye, and the lens. The aqueous humor is secreted continuously by the ciliary body around the lens leading to a constant flow of aqueous humor from the ciliary body to the eye's front chamber. Eye pressure is the result of the balance between the production of aqueous and its exit through the trabecular meshwork (major route) or uveal scleral outflow (minor route).

Glaucoma is grossly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous humor from the anterior chamber of the eye. Open-angle glaucoma is any glaucoma in which the angle of the anterior chamber remains open, but the exit of aqueous through the trabecular meshwork is diminished. The exact cause for diminished filtration is unknown for most cases of open-angle glaucoma.

Primary open-angle glaucoma is the most common of the glaucomas, and it is often asymptomatic in the early to moderately advanced stage. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

Current therapies for glaucoma are directed at decreasing intraocular pressure and include drug and surgical treatments. Drug therapy includes topical ophthalmic drops or oral medications that reduce the production or increase the outflow of aqueous. Drug therapies for glaucoma are sometimes associated with significant side effects, such as headache, blurred vision, allergic reactions, death from cardiopulmonary complications, and potential interactions with other drugs.

When drug therapy fails, surgical therapy is used. Surgical therapy for open-angle glaucoma consists of laser trabeculoplasty, trabeculectomy, and implantation of aqueous shunts after failure of trabeculectomy or if trabeculectomy is unlikely to succeed. Approximately 100,000 trabeculectomies are performed on Medicare-age patients per year in the United States. The surgical techniques that have been tried and practiced are goniotomy/trabeculotomy and other mechanical disruptions of the trabecular meshwork, such as trabeculopuncture, goniophotoablation, laser trabecular ablation, and goniocurretage. Therefore, there is a great clinical need for a method of treating glaucoma that is faster, safer, and less expensive than currently available modalities.

### SUMMARY OF THE INVENTION

The present invention relates to new compositions and methods for the treatment of disorders of the eye or skin, and more particularly, to the characterization and therapeutic use of modulators of intracellular calcium channel signaling in cellular physiology. Unlike other methods for the treatment of ocular degeneration that include surgery and the like, the present invention have the advantage that it is non-invasive, it has increased ease of use in application and therapeutics, it has an impact on a large number of patients affected worldwide and may also be used in cosmetics and/or cosmoceutical industry.

More particularly, the present invention includes compositions and methods to treat glaucoma, retinal neurodegenerative disease or macular degeneration modulating intracellular calcium concentrations when administered to a subject, the composition having an effective amount of an N-acylethanolamine (NAE) adapted for ocular administration.

The present invention includes a composition and methods to treat glaucoma, retinal neurodegenerative disease or macular degeneration by modulating intracellular calcium concentrations when administered to a subject, the composition comprising an effective amount of an N-acylethanolamine adapted for ocular delivery. The composition may also include a pharmaceutically acceptable carrier is selected for transcorneal delivery and include an effective amount of N-acylethanolamine that is between about 0.01 and 500 mg/ml or even between about 1 and 50 mg/ml. The N-acylethanolamine may be selected from the group consisting of N-acylethanolamine 12:0, 14:0, 16:0, 18:0, 18:2 and combinations thereof and may even be plant-derived. The composition maybe adapted for application to skin following a surgical procedure, for cosmetic use, for treatment of hair, for treatment of nails, for acute or chronic topical ocular application, for keratoplasty, for laser eye surgery, for induced cornea damage and combinations thereof.

In another embodiment, the composition to treat damage to skin by modulating intracellular calcium concentrations when administered to a subject, the composition with an effective amount of an N-acylethanolamine adapted for topical delivery. The composition may be adapted for topical delivery in a patch, medipad, ointment or cream. In some cases, the N-acylethanolamine is dissolved in water, saline or a lipophilic solution that is suitable for transdermal administration and/or dissolved in a lipophilic carrier suitable for topical administration.

The present invention also includes methods for treating glaucoma, retinal neurodegenerative disease or macular degeneration by administering to a subject in need thereof a composition comprising a pharmaceutically effective amount of an N-acylethanolamine. The method may include a treatment with a pharmaceutically acceptable carrier adapted for ocular delivery at, e.g., 0.1 and 50 mg/ml or even between about 1 and 10 mg/ml N-acylethanolamine. The N-acylethanolamine may be a N-acylethanolamine 12:0, 14:0, 16:0, 18:0, 18:2 and combinations thereof, which may be synthetic and/or plant-derived. The administration of the composition may be carried out once or even for a period of at least about 3 days, administered one or more times daily over a predetermined period, or administered as requested or required by a medical professional.

Another embodiment is a method for treating physical damage to the skin by administering to a subject in need thereof a composition comprising an effective amount of a plant-derived N-acylethanolamine adapted for topical delivery, e.g., before, during or after skin damage and for 1, 4, 8, 24, or even 48 hours after the occurrence of skin damage.

The present invention also includes a compound to treat glaucoma, retinal neurodegenerative disease or macular degeneration comprising the following formula: 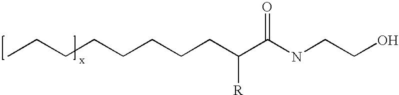
where x is 1, 2, 3, 4, 5, 6 or more;
and R is an alkyl, an aminoethanol or an aminoalcohol; and enantiomers thereof.

The present invention also includes a compound to treat skin by modulating intracellular calcium concentrations when administered to a subject comprising the following formula: 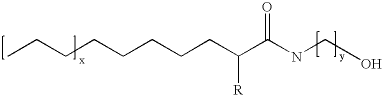
where: x is 1, 2, 3, 4, 5, 6;
where: y is 1, 2, 3, 4, 5, 6;
where R is an alkyl, an aminoethanol or an aminoalcohol; and enantiomers thereof.

The present invention also includes compositions and methods for treating a target skin site following a surgical procedure, for cosmetic use, for treatment of hair, for treatment of nails, for acute or chronic topical ocular application, for keratoplasty, for laser eye surgery, for induced cornea damage and combinations thereof, by administering to a subject in need thereof a composition adapted for administration at the site comprising a pharmaceutically effective amount of an N-acylethanolamine.

Depending on the extent of prevention or therapy, the composition may be carried out over a period of at least about 3, 7, 14 days or more, whether before, during or after the appearance or concern over the disease or condition that is to be treated. For example, the composition may be administered one or more times daily over a predetermined period. Examples of conditions that may be treated include a wide range of degenerative conditions of the eye and/or skin that results from changes in the level or extent of intracellular calcium channel signaling in a human or other mammal.

### BRIEF DESCRIPTION OF THE DRAWINGS

For a more complete understanding of the features and advantages of the present invention, reference is now made to the detailed description of the invention along with the accompanying figures and in which:

FIG. 1 is a mouse retina that shows no retinal ganglion cell death (measured by TUNEL staining; green) when vehicle controls of excitotoxic stimulation with glutamate are administered (top panels). Excitotoxic stimulation of the retina with glutamate results in death of retinal ganglion cells when vehicle controls of NAE treatments are administered (dying cells labeled with TUNEL staining; green; bottom panels);

FIG. 2 shows the effect of the treatment of mouse retina with NAE 18:2 alone;

FIG. 3 shows the effect of a 6 hour pre-incubation with vehicle (A), 20 μM (B), 40 μM (C), 80 μM (D), and 120 μM NAE 18:2 (E) prior to excitotoxic stimulation with glutamate and measured by TUNEL staining (green) shows retinal ganglion cell death when the vehicle control is administered (A) and dose dependent reduction of cell death with increasing NAE concentrations (B-E);

FIG. 4 shows the effect of co-application of vehicle (A) or 20 μM NAE 12:0 (B) with excitotoxic glutamate stimulation was combined with detection of markers for cell death (caspase-3; green) and for retinal ganglion cells (Thy-1.2; red);

FIG. 5 is a graph that shows mouse primary fibroblasts were exposed to a 16 hour treatment with 30 μM tert-butyl hydrogenperoxide (BHP) in the presence or absence of NAE 18:2;

FIG. 6 is a graph that shows mouse primary fibroblasts were exposed to a 16 hour treatment with 30 μM tert-butyl hydrogenperoxide (BHP) in the presence or absence of NAE 16:0;

FIG. 7 is a graph that shows neuroprotection of retinal ganglion cells (RGC) in the Morrison model of glaucoma (chronic pressure-induced optic nerve damage; and

FIG. 8 is a graph that shows 6 hour pre-incubation with vehicle, 20 μM, 40 μM, 80 μM, and 120 μM NAE 18:2 prior to excitotoxic stimulation with glutamate and measured by TUNEL staining.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

All technical and scientific terms used herein have the same meaning as commonly understood by one of ordinary skill in the art to which this invention belongs, unless defined otherwise.

The method of the present invention is adapted for the treatment of glaucoma, retinal neurodegenerative disease or macular degeneration, in which “adapted for” is used to describe those compounds that are specifically selected and prepared for the method of the present invention and includes, without limitations, e.g., a compositions and method for the treatment of ill patients who must meet stringent requirements to be included as patients with glaucoma, retinal neurodegenerative disease or macular degeneration. The present invention may also be applied to the skin following a surgical procedure, for cosmetic use, for treatment of hair, for treatment of nails, for acute or chronic topical ocular application, for keratoplasty, for laser eye surgery and/or for cornea damage (accidental or induced). In addition, pharmaceutically effective doses of the mixture are discussed, e.g., “pharmaceutically active” is construed in the context of the treatment of glaucoma, retinal neurodegenerative disease or macular degeneration, applied to the skin following a surgical procedure, for cosmetic use, for treatment of hair, for treatment of nails, for acute or chronic topical ocular application, for keratoplasty, for laser eye surgery and/or for cornea damage (accidental or induced).

As used herein, the term “effective amount” is used to describe the amount of active agent that modulates the release of calcium by intracellular calcium channels in cells of the skin or the eye. Depending on the intracellular calcium channel isoform, one or more NAEs may be administered to the patient to modify the intracellular calcium response in the eye and/or the skin. As used herein the term “lipophilic pharmacophor” is used to describe a plant protective agent that is used as a carrier for the NAE. The NAE may be provided in a carrier, e.g., a pharmaceutically effective carrier that aids in the delivery of the NAE.

As used herein, the term “subject” is intended to include living organisms in which certain conditions as described herein can occur. Examples include humans, monkeys, cows, sheep, goats, dogs, cats, mice, rats, and transgenic species thereof. In one embodiment, the subject is a primate, e.g., a human. Other examples of subjects include experimental animals such as mice, rats, dogs, cats, goats, sheep, pigs, and cows. The experimental animal may be an animal model for a disorder, e.g., a transgenic mouse with an glaucoma, retinal neurodegenerative disease or macular degeneration, applied to the skin following a surgical procedure, for cosmetic use, for treatment of hair, for treatment of nails, for acute or chronic topical ocular application, for keratoplasty, for laser eye surgery and/or for cornea damage (accidental or induced) or a normal animal or cells from an animal treated to have a “disease-like” condition, exposure to UV or other rays, surgery, or chemically-induced conditions.

The NAEs may be administered, e.g., cutaneous, topical, ocular and/or subcutaneous. Depending on the route of administration, the active compound may be coated in a material to protect the compound from the action of acids and other natural conditions which may inactivate the compound. When administering the therapeutic compound it may be necessary to coat the compound with, or co-administer the compound with, a material to prevent its inactivation as is well known in the art. For example, the therapeutic compound may be administered to a subject in an appropriate carrier, for example, liposomes, or a diluent. Pharmaceutically acceptable diluents include, e.g., lotions, saline and aqueous buffer solutions. Liposomes include water-in-oil-in-water emulsions as well as conventional liposomes. Dispersions may be prepared in glycerol, liquid polyethylene glycols, and mixtures thereof and in oils. These preparations may contain a preservative to prevent the growth of microorganisms depending on the ordinary conditions of storage and use.

Pharmaceutical compositions suitable for topical administration include, e.g., sterile aqueous solutions (where water soluble) or dispersions and sterile powders for the extemporaneous preparation of sterile injectable solutions or dispersion. The composition may be sterile and fluid to the extent for delivery. Generally, the compounding (pharmaceutically acceptable carrier and/or salt form (if any)) must be stable under the conditions of manufacture and storage and must be preserved against the contaminating action of, e.g., microorganisms such as bacteria and fungi. A carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, e.g., by the use of a coating such as lecithin, by the maintenance of the required particle size in the case of dispersion and by the use of surfactants. The composition may also include antibacterial and antifungal agents, for example, parabens, chlorobutanol, phenol, ascorbic acid, thimerosal and the like. In many cases, it will be preferable to include isotonic agents, for example, sugars, sodium chloride, or polyalcohols such as mannitol and sorbitol, in the composition. Prolonged absorption of the injectable compositions may be achieved by including an agent that delays absorption, for example, aluminum monostearate or gelatin.

Sterile solutions for use with the present invention may be prepared by incorporating the NAEs of the present invention at an appropriate amount and in an appropriate solvent with one or a combination of ingredients described above followed by filtered sterilization. Generally, dispersions may be prepared by incorporating the therapeutic compound into a carrier that includes a basic dispersion medium and the required other ingredients from those enumerated above. In the case of sterile powders, drops, serums or lotions, the compound may be prepared in solid form by, e.g., vacuum drying or freeze-drying, which yields a powder of the active ingredient (i.e., the therapeutic compound) plus any additional desired ingredient from a previously sterile-filtered solution thereof. The percentage of the NAEs in the final preparations may, of course, be varied to deliver the amount of NAE in a therapeutically useful composition such that a suitable dosage is obtained.

Dosage unit form as used herein refers to physically discrete units suited as unitary dosages for the subjects to be treated, i.e., each unit includes a predetermined quantity of NAE(s) calculated to produce the desired therapeutic effect in association with the required pharmaceutical carrier. The specifications for the dosage unit of the NAEs of the present invention are dictated by, and directly dependent on, e.g., the unique characteristics of the NAE(s) and the particular therapeutic effect to be achieved and (b) the limitations inherent in the art of compounding such an NAE(s) for the treatment of a selected condition in a subject.

Active compounds are administered at a “therapeutically effective dosage” are those sufficient to treat a condition associated with a “condition” in a “subject.” For example, a “therapeutically effective dosage” reduces the amount of symptoms of the condition in the infected subject by at least about 20%, at least about 40%, at least about 60%, and at least about 80% relative to untreated subjects. For example, the efficacy of a compound can be evaluated in an animal model system that may be predictive of efficacy in treating the disease in humans, such as the model systems described herein and/or that are known to those of skill in the art.

As used herein, the term “cosmeceutical” refers to a product, typically non-prescription, that is used in the cosmetic industry and that produces a measurable structural change in the skin and/or a mucous membrane.

The NAEs of this invention can be incorporated into various types of ophthalmic formulations for delivery to the eye (e.g., topically, intracamerally, or via an implant). The compounds are often incorporated into topical ophthalmic formulations for delivery to the eye, e.g., with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound.

The ophthalmic solution may also include an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents may also be used, including, but not limited to, gellan and xanthan gum. To prepare sterile ophthalmic ointment formulations, the NAEs may be combined with a preservative in an appropriate vehicle, such as, mineral oil, liquid lanolin, or white petrolatum. A sterile ophthalmic gel formulation may be prepared by suspending the active ingredient in a hydrophilic base prepared from the combination of, e.g., carbopol-974, or the like, according to the published formulations for analogous ophthalmic preparations; preservatives and tonicity agents can be incorporated.

For use in the eye and increase patient compliance, the compounds are generally formulated as topical ophthalmic suspensions or solutions with a pH of about 5 to 8. The compounds are normally contained in an inert carrier or diluent in an amount 0.01% to 5% by weight or even in an amount of 0.25% to 2% by weight. For topical administration 1 to 2 drops of these formulations would be delivered to the surface of the eye 1 to 4 times per day according to the discretion of a skilled clinician.

The NAEs may also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., briazolamide and dorzolamide), nipradolol, iopidine and brimonidine, miotics (e.g., pilocarpine and epinephrine) and/or prostaglandin analogs (e.g., latanoprost, travaprost, unoprostone, and the like.

The NAEs of the present invention may also be included in or prepared as part of a modified topical skin composition, that is includes a foundation for a cosmetic or cosmeceutical material that includes an effective amount of the NAE sufficient to treat a skin condition such as treating a target skin site following a surgical procedure, for cosmetic use, for treatment of hair, for treatment of nails, for acute or chronic topical ocular application, for keratoplasty, for laser eye surgery, for induced cornea damage and combinations thereof, wherein the composition is provided in liquid, semi-liquid, semi-solid, gel pr solid form and readily absorbed by an epidermal layer of mammalian skin to permit passage of infrared energy through the epidermal layer with reduced absorption of said energy by the epidermal layer. The cosmetic or cosmeceutical material is generally in the form of liquid emulsions (lotions) or thicker emulsions (creams).

The NAEs may be incorporated in an effective amount into a cosmetic or cosmeceutical material in the form of lotions, creams, solutions, suspensions, anhydrous salves, sticks, gels, emulsions, ointments, plasters, patches, films, tapes or dressing preparations, all of which are known to those of ordinary skill in the art of topical skin formulations and preparations.

Previously, the inventors have identified, characterized and used various NAE molecular species in higher plants, and has developed procedures for the routine use in U.S. patent application Ser. No. 10/840,449, incorporated herein by reference. Studies were conducted to demonstrate that N-acylethanolamines, e.g., from plant tissues have a protective effect and to develop and implement novel therapies for neurological disorders. These studies support ongoing interests in the physiological role of NAEs in plant cells, but also form the basis for accurate quantification of these metabolites in natural products for the purposes of standardization. It is interesting that different plant tissue sources contain different NAE species, with seeds being particularly rich in NAE 18:2. The following is the basic structure of the base structure of the NAEs of the present invention,

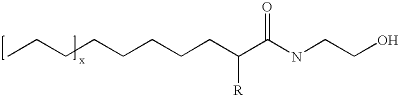

where: y is 1, 2, 3, 4, 5, 6 or more; and r is an alkyl, e.g., H, CH₃, CH₂CH₃, CH₂CH₂CH₃, CH₂CH₂CH₂CH₃, an aminoethanol or an aminoalcohol and enantiomers thereof, etc.

Yet another structure of an NAE of the present invention is: 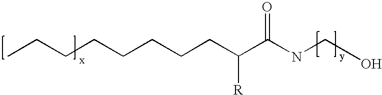
where: x is 1, 2, 3, 4, 5, 6; y is 1, 2, 3, 4, 5, 6; and R is an alkyl, e.g., H, CH₃, CH₂CH₃, CH₂CH₂CH₃, CH₂CH₂CH₂CH₃, an aminoethanol or an aminoalcohol and enantiomers thereof.

Briefly, the intracellular calcium channel modulators of the present invention may include at the C-2 position of the parent NAE, e.g., a small alkyl (Me, Et, Propyl, Butyl) group, aminoethanols and aminoalcohols, including enantiomers thereof. For example, the aminoethanol group in NAE may be replaced with a different aminoalcohol. Such alternative head groups have been reported for anandamide analogues (Khanolkar, A. D., Abadji, V., Lin, S., Hill, A. G., Taha, G., Abouzid, K., Meng, Z., Fan, P., & Makriyannis, A. Head group analogs of arachidonylethanolamide, the endogenous cannabinoid ligand. J Med Chem, 39, 4515-19 (1996)), relevant portions incorporated herein by reference. In addition to synthetic sources of NAEs, another source are extracts from plant materials have been prepared which, depending on the species and tissue source, contained a varied composition of bioactive NAEs. Synthetic and/or modifications of NAEs from extracts may also be generated, as such, these enantiomers and preparations of R and/or S enantiomers and mixtures thereof may be used with the present invention.

This invention is a new use and an improvement to an existing product, its effects and mode of application. This disclosure is to declare the discovery of novel functions of N-acylethanolamines in the modulation of intracellular calcium signaling and in cellular physiology. N-acylethanolamine 12:0, related compounds and precursors (here referred to as N-acylethanolamines or NAEs) specifically modulate the activity of intracellular calcium channels and thereby influence the calcium homeostasis inside of cells. This new knowledge allows us to pharmacologically manipulate intracellular calcium signaling a process that is relevant for physiological and pathophysiological functions of cells, including, but not limited to Alzheimer's disease, stroke, traumatic head and spinal cord injury, glaucoma, retinal ischemia, cardiac failure and ischemia, cancer.

One embodiment of this invention is to administer NAEs locally, topically, via a transdermal or transcorneal route or in general to the exterior surface of the body. This application of NAEs to protection cells from adverse external or intrinsic effects, cellular degeneration and death can happen prior to, during, or after the observation of symptoms of diseases involving perturbation of the intracellular calcium homeostasis and to prevent the progression of the diseases or to prevent their occurrence.

It is disclosed herein that NAEs can exert their effects also through local and topical administration via a transdermal or transcorneal route or in general by application to the exterior surface of the body. In addition, we expand the number of potential diseases for which NAEs can provide prevention of disease progression or of their occurrence to glaucoma, retinal neurodegenerative disease such as macular degeneration as well as mechanical and other physical damage to the skin and other exterior surfaces or parts of the body. This provides a significant advantage due to the increased ease of use and application as well as due to the expanded scope of potential applications.

Material and methods. Organotypic retina cultures were prepared and subjected to glutamate-induced neurotoxicity as described previously by Hua Xin, Jo-Ann S. Yannazzo, R. Scott Duncan, Elaine V. Gregg, Meharvan Singh, and Peter Koulen. Journal of Neuroscience Methods. 2007 Jan. 15;159(1):35-42. Epub 2006 Jul. 31., A novel organotypic culture model of the postnatal mouse retina allows the study of glutamate-mediated excitotoxicity, relevant portions incorporated herein by reference.

Animal preparation and retina explant culture. To avoid contamination, instruments were heat sterilized at 250° C. for 3 min, and surgical instruments were heated repetitively during tissue preparation. C57BL/6 mice at different postnatal ages (P10-P14) were euthanized by over-exposure to CO₂ and decapitated and the freshly enucleated eyes were immersed in cold Hank's Balanced Salt Solution (HBSS) (HyClone Inc., Logan, Utah) under sterile conditions afforded by a laminar flow hood. After dissection of the retina, remaining vitreous and retinal pigment epithelium (RPE) were carefully removed from the neural tissue. After introducing incisions in the shape of a Maltese cross to flatten out the retina tissue, the retina was transferred to poly-d-lysine/laminin coated glass coverslip (BD Bioscience, Bedford, Mass.) with the ganglion cell layer facing the coverslip. The retina was allowed to attach to the coverslip for 30 min at room temperature. Then the coverslips with the retina tissue were transferred to six-well plates and 20 μl of culture medium (Neurobasal-A Medium, Gibco, Carlsbad, Calif. with 2% DHS (donor horse sera), B27 supplement and Penicillin-Streptomycin-Fungizone) was added to each culture. During incubation at 37° C. with 95% air/5% CO₂, medium was exchanged every other day.

Glutamate treatment/neurotoxic insult. Glutamate (Sigma, St. Louis, Mo.) at different concentrations in culture medium (10 μM, 50 μM and 100 μM with 0.5% Triton X-100) was added to ex vivo cultures after 7 days in culture and incubated at 37° C. with 95% air/5% CO₂ overnight (18 h). The experimental conditions and especially the concentrations and extended incubation times for glutamate described in the present paper were chosen to compensate for glutamate uptake by retinal glial cells and metabolization by glutamine synthetase, which is typical for ex vivo whole-mount and mixed glia-neuron culture systems. The method presented in Haberecht et al. (1997) employed the receptor-specific glutamate agonist NMDA, which is not metabolized as readily as glutamate and leads to faster excitotoxic neuronal degeneration and death. A neurotoxic insult using glutamate was chosen in the present paper to model various relevant actions associated with glutamate-induced cell death under patho-physiological conditions of high extracellular glutamate concentrations, such as the activation of multiple receptor types and the effects on the cellular redox potential mediated by interference with cysteine transport and glutathione synthesis. Nevertheless as described above, the model also allows other pharmacological and toxicological interventions, assessments and manipulation due to its experimental accessibility as an ex vivo system.

Briefly, after 7 days in culture, the ex vivo cultured retina were divided in four groups: (1) Glutamate-treated group: exposure to 100 μM glutamate for 16 hours; (2) Vehicle-treated group: exposure to vehicle in amounts equivalent to NAE treated group for 16 hours; (3) Glutamate+Vehicle-treated group: exposure to 100 μM glutamate and to vehicle in amounts equivalent to NAE treated group for 16 hours; and (4) Glutamate+NAE-treated group: exposure to 100 μM glutamate and to NAE for 16 hours (NAE used at 20, 40, 80, 120 μM; vehicle for NAE 18:2, ethanol; for NAE 12:0, water). Compounds and controls were administered either at the same time or NAEs/respective vehicle were added 6 hours prior to addition of the glutamate insult.

The histological analysis and quantification of neuroprotection by TUNEL assay was done as described previously by Hua Xin, Jo-Ann S. Yannazzo, R. Scott Duncan, Elaine V. Gregg, Meharvan Singh, and Peter Koulen. Journal of Neuroscience Methods. 2007 Jan. 15;159(1):35-42. Epub 2006 Jul. 31, A novel organotypic culture model of the postnatal mouse retina allows the study of glutamate-mediated excitotoxicity, relevant portions incorporated herein by reference.

TUNEL assay/detection of apoptosis. Apoptotic cells in organotypic cultures of the retina after glutamate treatment were detected with the DeadEnd Fluorometric TUNEL System (Promega, Madison, Wis.) following the manufacturer's instruction. Briefly, retina cultures attached to coverslips were fixed in 4% paraformaldehyde for 25 min at 4° C., washed in PBS and permeabilized in PBS containing 1% Triton X-100 for 30 min at room temperature. After PBS wash, the retina cultures were covered with 20 μl of equilibration buffer for 10 min at room temperature. After removing excess liquid, the tissue was covered with 20 μl TdT enzyme buffer and incubated at 37° C. for 60 min. To stop the reaction, 2X SSC solution was added to the retina cultures for 15 min and tissue was washed in PBS for 5 min at room temperature. Tissue was mounted on microscope slides as whole-mounts with Prolong Gold antifade reagent mounting medium containing 1.5 μg/ml DAPI (Molecular Probes, Eugene, Oreg.). The samples were analyzed with standard epi-fluorescence microscope and digital microphotography (SimplePCI, Compix Inc., Image Systems, Sewicklely, Pa.). Stained TUNEL-positive RGCs were then counted in photographs of one microscopic field of each retina explant. Controls included cultures without glutamate treatment and a negative control with glutamate treatment but without TdT enzyme incubation. In addition to staining of whole-mount cultures, the TUNEL assay was also repeated with sections of retina explant cultures.

Cryostat section of retina ex vivo culture and immunocytochemical staining of retinal cells. Retina cultures were fixed in 4% paraformaldehyde overnight at 4° C. After removal of cultures from the coverslips and embedding in OCT compound (Sakura Finetek USA Inc., Torrance, Calif.), retina cultures were sectioned vertically (12 μm thickness) on a cryostat microtome. In order to view the architecture of the retina cultures, standard Hematoxylin-Eosin (HE) staining was carried out. Retinal ganglion cells were detected with standard indirect immuno-fluorescence for Neurofilament68 kDa immunoreactivity (Chemicon, Temecula, Calif.). Briefly, sections were permeabilized in blocking buffer (10% normal goat serum (NGS), 2% BSA, 0.5% Triton X-100 in PBS) for 60 min at room temperature, and then incubated with the primary antibody directed against Neurofilament-68 KDa diluted 1:500 in incubation buffer (5% NGS, 2% BSA, 0.5% Triton X-100 in PBS) overnight at 4° C. Immunoreactivity was detected with FITC conjugated goat anti-rabbit IgG secondary antibody diluted 1:1000 in incubation buffer for 90 min at room temperature. Sections were mounted with Prolong+DAPI and were analyzed with standard epi-fluorescence microscope and digital microphotography (SimplePCI). Retinal bipolar, horizontal, amacrine, photoreceptor and glial cells were also localized to further characterize the retinal cytoarchitecture. Vertical sections of mouse retina explants were stained with antibodies directed against Protein Kinase C alpha (PKC) (Chemicon, Temecula, Calif.), Calbindin (Chemicon), GABA (Sigma), all with a 1:500 dilution, respectively; Rhodopsin (Chemicon) diluted 1:80, and glial fibrillary acidic protein (GFAP) (Biomeda, Foster City, Calif.) diluted 1:1000.

The immunohistological analysis and quantification of neuroprotection by immunohystochemistry assays was done as described previously by Mafe, Oloruntoyin; Gregg, Elaine; Medina-Ortiz, Wanda; Koulen, Peter. Journal of Neuroscience Research Dec;84(8):1750-1758, ‘Localization of inositol 1,4,5-trisphosphate receptors in mouse retinal ganglion cells.’

Immunohistochemistry. Immunohistochemistry was carried out as described previously (Koulen and Brandstatter, 2002; Kaja et al., 2003). Retinas were removed from the eye cups and tissue was immersion fixed in 4% paraformaldehyde in phosphate buffer (0.1 M PBS, pH 7.4) for 30 min. Vertical cryosections of mouse retina tissue were cryoprotected by infusion with 10, 20, and 30% sucrose and then frozen. Cryosections (12 lm) of retinal tissue were used in all experiments. immunohistochemistry was carried out using custom IP3R Types 1, 2 and 3 antibodies (diluted 1:1,000).

Immunocytochemistry. Immunocytochemistry was carried out essentially as described by Leite et al. (2003). After culture for 14 days, RGCs were fixed using 4% wt/vol paraformaldehyde (PFA) in phosphate buffer solution (PBS 0.1 M; pH 7.4) for 30 min. Immunocytochemical labeling was carried out by indirect fluorescence method. Nonspecific binding sites were blocked by incubating the cells in PBS (0.1 M, pH 7.4) containing 10% (vol/vol) normal goat serum (NGS), 1% (vol/vol) BSA, and 0.05% Triton X-100 for 1 hr. Primary and secondary antibodies were diluted in PBS containing 3% NGS, 1% BSA, and 0.05% Triton X-100. IP3R Types 1, 2, and 3 antisera were used at a dilution of 1:1,000 and RGCs were incubated overnight at 48 C. Binding sites of the primary antibodies were revealed by secondary antibodies. Control experiments in which the primary antibodies were omitted showed no specific staining. Immunofluorescence labeling was examined and photographed using the Olympus IX70, (Olympus, Japan), Hamamatsu ORCA-ER (Hamamatsu, Japan), Lambda DG-4 Ultra High Speed Wavelength Switcher with appropriate filter sets (Sutter Instrument Company, Novato, Calif.), and Simple PCI Imaging Software v. 5.2 (Compix Inc., Imaging Systems/Hamamatsu Photonics Management Corporation, Bridgewater, N.J.).

Immunopanning. Retinas were dissociated enzymatically as described earlier. A modified immunopanning procedure was carried out essentially as described by Barres et al. (1988) and Otori et al. (2003). To prevent nonspecific binding of cells to the panning plates, the plates were washed initially with 2 ml of sterile 0.1 M PBS with 0.1% BSA. Each of the two plates was incubated with 5.0 ml of PBS with OX-42 (1:25) and 5.0 ml of PBS with Thy 1.2 (1:500) and left overnight at 48C. Retinal suspensions were then incubated in OX-42 for 30 min at room temperature in the dark. Pre-incubation with OX-42 was important to remove microphages and microglia that would otherwise interact with anti-Thy 1.2 antibodies. Suspensions were moved every 10 min to ensure access of all cells to the surface of the coating area. Non-adherent cells were removed and placed in Thy 1.2-coated plates for 30 min at room temperature. After 30 min, non-adherent cells were removed and plates were washed gently two times with TF medium. Finally, adherent cells on Thy 1.2-coated plates were removed either by scraping the plates or with 20011 of 0.125% trypsin (HyClone, Logan, Utah.). Cells were washed three times with TF medium and centrifuged at 1,000 rpm for 10 min and the pellet obtained was stored at 208 C or 808 C for Western blot analysis and some were re-suspended in 400 11 TF medium, seeded, and cultured on 12-mm glass coverslips as described previously. Trypsin was inactivated by adding an equal volume of a trypsin inhibitor solution (chicken egg white; Sigma).

Immunoblotting Analysis. RGCs (obtained from immunopanning as described previously) were homogenized in a buffer containing 250 mM sucrose, 5 mM HEPES, 100 mM EGTA (Sigma), with a mixture of protease inhibitors (10 lg/ml trypsin, 1 mM pepstatin, 10 mM leupeptin, and 2 mg/ml aprotinin). Protein samples (15-20 lg) were loaded and separated using 4-15% gradient gels. After electrophoresis, the gels were equilibrated in transfer buffer for 1 hr at 60 V. Before immunoblotting, the PVDF membranes were blocked with 2.5% BSA (in 0.1 M PBS, pH 7.4 containing 0.05% Tween 20) for 1 hr. Membranes were incubated with IP3R Types 1, 2, and 3 at 48 C overnight, then washed two times with washing solution (0.1 M PBS, pH 7.4 containing 0.05% Tween 20) and incubated with secondary antibodies (1:2,000) for 1 hr. Membranes were developed to visualize protein bands using Super-signal West Dura Extended Duration Substrate Kit.

FIG. 1 is a mouse retina that shows no retinal ganglion cell death (measured by TUNEL staining; green) when vehicle controls of excitotoxic stimulation with glutamate are administered (top panels). Excitotoxic stimulation of the retina with glutamate results in death of retinal ganglion cells when vehicle controls of NAE treatments are administered (dying cells labeled with TUNEL staining; green; bottom panels). Blue: DNA marker labeling cell nuclei; gray: differential interference contrast images.

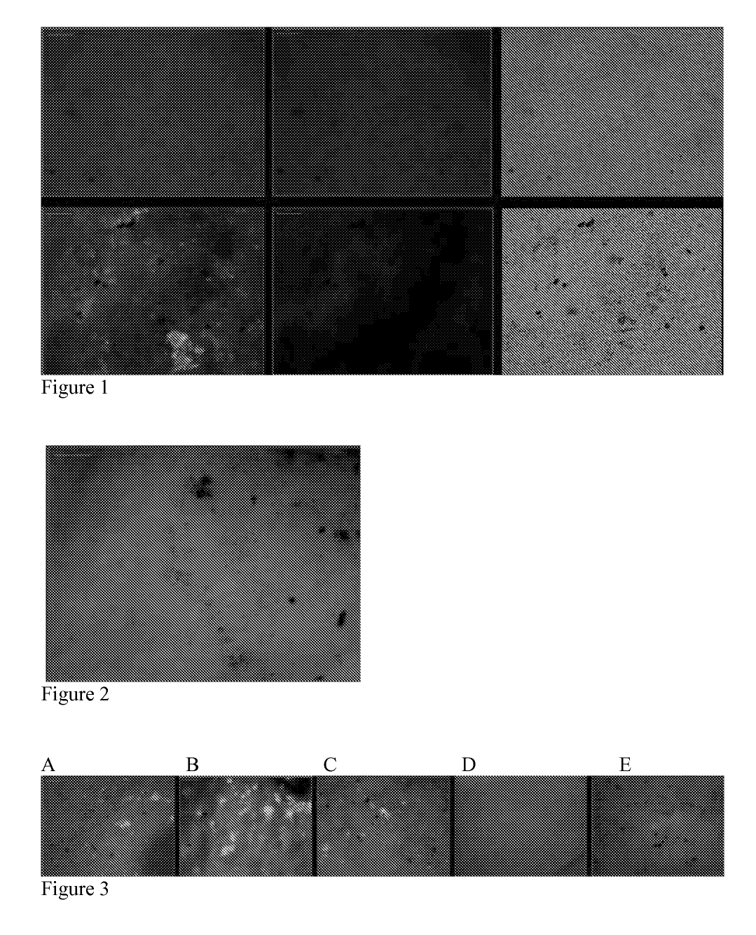

FIG. 2 shows the effect of the treatment of mouse retina with NAE 18:2 alone shows no retinal ganglion cell death other than background staining (measured by TUNEL staining; green).

FIG. 3 shows the effect of a 6 hour pre-incubation with vehicle (A), 20 μM (B), 40 μM (C), 80 μM (D), and 120 μM NAE 18:2 (E) prior to excitotoxic stimulation with glutamate and measured by TUNEL staining (green) shows retinal ganglion cell death when the vehicle control is administered (A) and dose dependent reduction of cell death with increasing NAE concentrations (B-E).

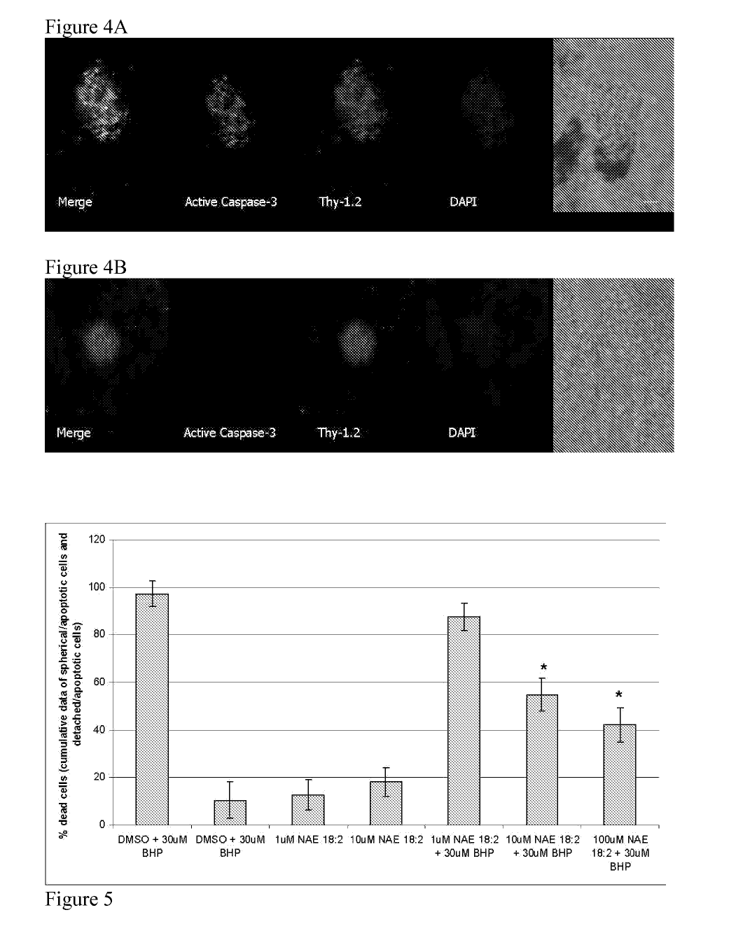

FIG. 4 shows the effect of co-application of vehicle (A) or 20 μM NAE 12:0 (B) with excitotoxic glutamate stimulation was combined with detection of markers for cell death (caspase-3; green) and for retinal ganglion cells (Thy-1.2; red). Activation of caspase-3 was not detected when cell were incubated with NAE 12:0 (B) when compared to vehicle (A). Blue: DNA marker labeling cell nuclei.

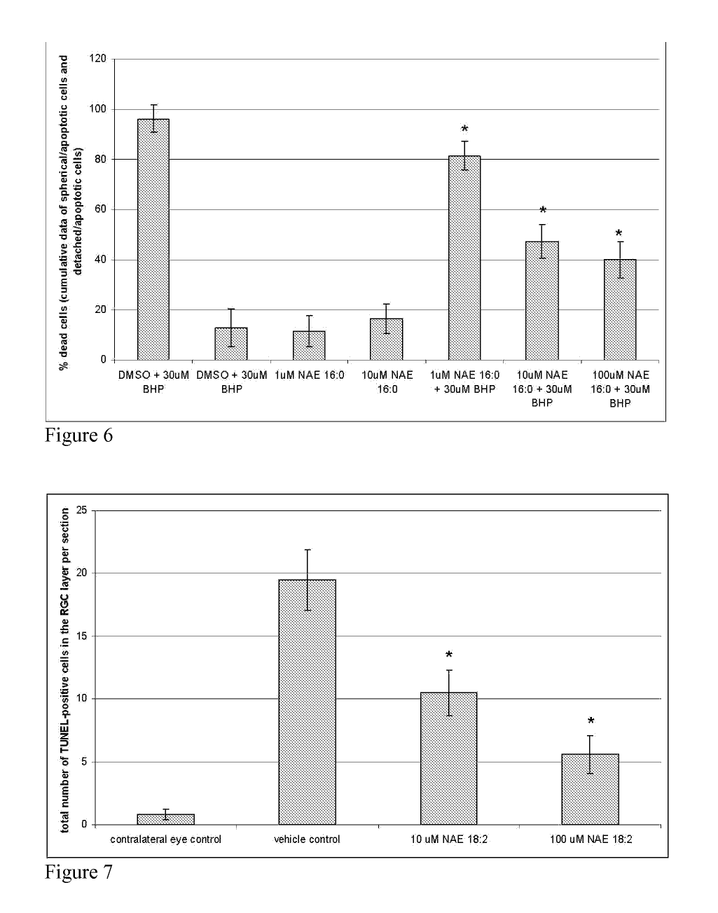

As shown in FIGS. 5 and 6, Poly-L-lysine-coated 12 mm coverslips are used for plating of mouse primary skin fibroblasts. Plated cells were grown in DMEM+5% BGS (medium) to a confluency of about 50%. NAEs were diluted in medium to a final concentration of 1-100 μM and warmed to 37 deg C. The chemical insult, addition of 30 μM tBHP was combined with the addition of NAEs and vehicle controls and cells were incubated for 16 hours. After incubation cells were fixed with 4% aldehyde solution for histological analysis and quantification of cytoprotection by TUNEL assay as described above for FIGS. 1-4.

FIG. 5 is a graph that shows mouse primary fibroblasts were exposed to a 16 hour treatment with 30 μM tert-butyl hydrogenperoxide (BHP) in the presence or absence of NAE 18:2. The percentage of dead cells was measured as cumulative data of spherical/apoptotic cells and detached/apoptotic cells. Asterisks indicate statistically significant difference from vehicle control (t-test). Dose-dependent reduction of cell death with increasing NAE concentrations was observed.

FIG. 6 is a graph that shows mouse primary fibroblasts were exposed to a 16 hour treatment with 30 μM tert-butyl hydrogenperoxide (BHP) in the presence or absence of NAE 16:0. The percentage of dead cells was measured as cumulative data of spherical/apoptotic cells and detached/apoptotic cells. Asterisks indicate statistically significant difference from vehicle control (t-test). Dose-dependent reduction of cell death with increasing NAE concentrations was observed.

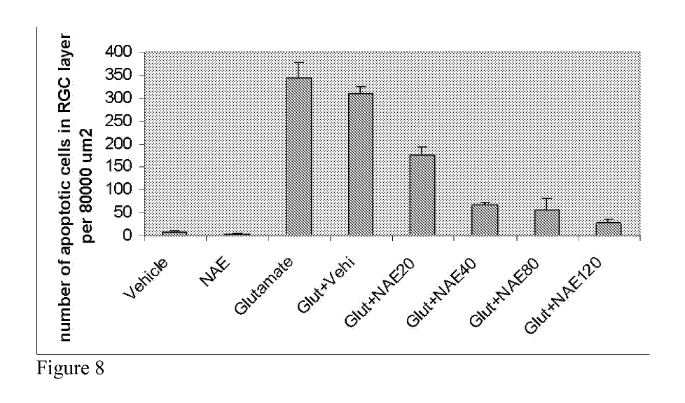

For FIGS. 7 and 8, the procedure that was used to elevate the rat IOP was as previously described by Morrison, J. C., et al. A rat model of chronic pressure-induced optic nerve damage. Exp Eye Res. 64, 85-96 (1997).

Briefly, female Brown Norway rats (Harlan, Indiana) with ovariectomy weighing between 160 and 200 g were used in this study and initially housed under standard 12-h light/12-h dark cycle and room temperature was maintained at 21° C. Surgery for IOP elevation was performed on anaesthetized rats [i.p. injection of a standard rat cocktail (1 ml/kg), consisting of a mixture of ketamine (5 ml of 100 mg/ml), xylazine (0.5 ml of 100 mg/ml) and acepromazine (1 ml of 10 mg/ml) and 0.5 ml of water)]. One eye (left side) of each animal was used for the intraocular pressure (IOP) elevation. 50 μl of 1.8 M hypertonic saline was injected into the episcleral vein using a micro glass needle with an injection pump. After stabilization, IOP measurements were taken with a Tono-Pen XL tonometer (Mentor, Norwell, Mass.) on conscious animals in the presence of the topical anaesthesia, proparacaine 0.1%. IOP was monitored twice a week for up to 10-14 days, once IOP was greater than 25% of contra lateral eye (right side) IOP values otherwise a second injection would be performed. Rats with elevated IOP were maintained for up to 30 days post-surgery with IOP monitoring twice a week. Following the experimentation period, all rats were euthanized by an i.p. injection of pentobarbital (120 mg/kg). After elevated IOP stabilization, rats were randomly divided into the same groups as described for FIGS. 1-4 (each group, n=4). 20% 2-hydroxypropyl-β-cyclodextrin was used as a vehicle. 10 μl of solutions were applied as eye drops.

TUNEL assay/detection of apoptosis. After rats were euthanized, the freshly enucleated eyes were immersed in Hank's Balanced Salt Solution (HBSS) (HyClone Inc., Logan, Utah.) and then were fixed in 4% paraformaldehyde overnight at 4° C. The eyes were embeded in OCT compound (Sakura Finetek USA Inc., Torrance, Calif.) and were sectioned vertically (12 μm thickness) on a cryostat microtome. Apoptotic cells in eye sections were detected with the DeadEnd Fluorometric TUNEL System (Promega, Madison, Wis.) following the manufacturer's instruction and as described above for FIGS. 1-4. Briefly, sections were fixed in 4% paraformaldehyde for 25 min at 4° C., washed in PBS and permeabilized in PBS containing 1% Triton X-100 for 30 min at room temperature. After PBS wash, the eye sections were covered with 20 μl of equilibration buffer for 10 min at room temperature. After removing excess liquid, the sections were covered with 20 μl TdT enzyme buffer and incubated at 37° C. for 60 min. To stop the reaction, 2×SSC solution was added to sections for 15 min and sections were washed in PBS for 5 min at room temperature. Sections were mounted on cover slips with Prolong Gold antifade reagent mounting medium containing 1.5 μg/ml DAPI (Molecular Probes, Eugene, Oreg.). The samples were analyzed with standard epi-fluorescence microscope and digital microphotography (SimplePCI, Compix Inc., Image Systems, Sewicklely, Pa.). Stained TUNEL-positive RGCs were then counted in photographs of each eye section. A negative control was conducted without TdT enzyme incubation.

FIG. 7 is a graph that shows neuroprotection of retinal ganglion cells (RGC) in the Morrison model of glaucoma (chronic pressure-induced optic nerve damage; injection of hypertonic solution into the episcleral vein leads to scarring of the trabecular meshwork and impedes aqueous humor outflow and results in elevated intraocular pressure (IOP). IOP was measured 2-3 times weekly and remained stably elevated after initial increase. An effect of treatment on IOP was not observed. Compounds were administered topically as eye-drops, 10 μl per eye, daily. Animals were sacrificed 19 days after initial IOP elevation; eyes were sectioned at 16 μm and processed for in situ TUNEL assay to determine the number of apoptotic/dying cells. Dose-dependent reduction of cell death with increasing NAE concentrations was observed. Asterisks indicate statistically significant difference from vehicle control (t-test).

FIG. 8 is a graph that shows 6 hour pre-incubation with vehicle, 20 μM, 40 μM, 80 μM, and 120 μM NAE 18:2 prior to excitotoxic stimulation with glutamate and measured by TUNEL staining shows retinal ganglion cell death when the vehicle control is administered and dose dependent reduction of cell death with increasing NAE concentrations. The following pairs were found to have statistically significant difference from each other with P<0.01 (t-test). The comparison between:
Vehicle vs. Glutamate
Vehicle vs. Glutamate+Vehicle
Vehicle vs. Glutamate+NAE20
Glutamate vs. Glutamate+NAE40
Glutamate vs. Glutamate+NAE80
Glutamate vs. Glutamate+NAE120

indicate that concentrations of 40 μM, 80 μM, and 120 μM NAE 18:2 reduce cell death back to control levels. The comparison between:
NAE vs. Glutamate
NAE vs. Glutamate+Vehicle
NAE vs. Glutamate+NAE20
NAE vs. Glutamate+NAE40

indicate that NAE 18:2 itself is not causing cell death. Finally, the comparison between:
Glutamate+Vehicle vs. Glutamate+NAE20
Glutamate+Vehicle vs. Glutamate +NAE40
Glutamate+Vehicle vs. Glutamate +NAE80
Glutamate+Vehicle vs. Glutamate +NAE120
Glutamate+NAE20 vs. Glutamate +NAE120

indicate that NAE 18:2 dose-dependently protects from cell death.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Berdyshev E V, Schmid P C, Krebsbach R J, Hillard C J, Huang C, Chen N, Dong Z, Schmid H H. Cannabinoid-receptor-independent cell signalling by N-acylethanolamines. Biochem J. 2001 Nov. 15;360(Pt 1):67-75.

Berdyshev E V, Schmid P C, Dong Z, Schmid H H. Stress-induced generation of N-acylethanolamines in mouse epidermal JB6 P+cells. Biochem J. 2000 Mar. 1;346 Pt 2:369-74.

Gray G M. Phosphatidyl-(N-acyl)-ethanolamine. A lipid component of mammalian epidermis. Biochim Biophys Acta. 1976 Apr. 22;431(1):1-8.

What is claimed is:

1. A composition to treat glaucoma, retinal neurodegenerative disease or macular degeneration by modulating intracellular calcium concentrations when administered to a subject, the composition comprising an effective amount of an N-acylethanolamine adapted for ocular delivery.

2. The composition of claim 1, further comprising a pharmaceutically acceptable carrier is selected for transcorneal delivery.

3. The composition of claim 1, wherein the effective amount of N-acylethanolamine is between about 0.01 and 500 mg/ml.

4. The composition of claim 1, wherein the effective amount of N-acylethanolamine is between about 1 and 50 mg/ml.

5. The composition of claim 1, wherein the N-acylethanolamine is selected from the group consisting of N-acylethanolamine 12:0, 14:0, 16:0, 18:0, 18:2 and combinations thereof.

6. The composition of claim 1, wherein the N-acylethanolamine is plant-derived.

7. The composition of claim 1, wherein composition is adapted for application to skin following a surgical procedure, for cosmetic use, for treatment of hair, for treatment of nails, for acute or chronic topical ocular application, for keratoplasty, for laser eye surgery, for induced cornea damage and combinations thereof.

8. A composition to treat damage to skin by modulating intracellular calcium concentrations when administered to a subject, the composition comprising an effective amount of an N-acylethanolamine adapted for topical delivery.

9. The composition of claim 8, wherein adapted for topical delivery comprising a patch, medipad, ointment or cream.

10. The composition of claim 8, wherein the N-acylethanolamine is dissolved in water, saline or a lipophilic solution that is suitable for transdermal administration.

11. The composition of claim 8, wherein the N-acylethanolamine is dissolved in a lipophilic carrier suitable for topical administration.

12. A method for treating glaucoma, retinal neurodegenerative disease or macular degeneration, the method comprising the step of administering to a subject in need thereof a composition comprising a pharmaceutically effective amount of an N-acylethanolamine.

13. The method of claim 12, further comprising a pharmaceutically acceptable carrier adapted for ocular delivery.

14. The method of claim 12, wherein the effective amount of N-acylethanolamine is between about 0.1 and 50 mg/ml.

15. The method of claim 12, wherein the effective amount of N-acylethanolamine is between about 1 and 10 mg/ml.

16. The method of claim 12, wherein the N-acylethanolamine is selected from the group consisting of N-acylethanolamine 12:0, 14:0, 16:0, 18:0, 18:2 and combinations thereof.

17. The method of claim 12, wherein the N-acylethanolamine is plant-derived.

18. The method of claim 12, in which the administration of the composition is carried out over a period of at least about 3 days.

19. The method of claim 12, wherein the composition is administered one or more times daily over a predetermined period.

20. The method of claim 12, wherein the subject is human.

21. A method for treating physical damage to the skin, the method comprising the step of administering to a subject in need thereof a composition comprising an effective amount of a plant-derived N-acylethanolamine adapted for topical delivery.

22. The method of claim 21, wherein the composition is administered before, during or after skin damage.

23. The method of claim 21, wherein the composition is administered within 1, 4, 8, 24, or even 48 hours after the occurrence of skin damage.

24. A compound to treat glaucoma, retinal neurodegenerative disease or macular degeneration comprising the following formula: 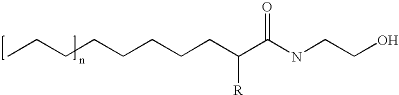
where: x is 1, 2, 3, 4, 5, 6 or more; and R is an alkyl, an aminoethanol or an aminoalcohol; and enantiomers thereof.

25. A compound to treat skin by modulating intracellular calcium concentrations when administered to a subject comprising the following formula: 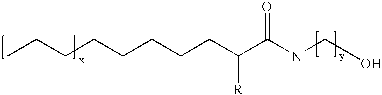
where: x is 1, 2, 3, 4, 5, 6;
where: y is 1, 2, 3, 4, 5, 6;
where R is an alkyl, an aminoethanol or an aminoalcohol; and enantiomers thereof.

26. A method for treating a target skin site following a surgical procedure, for cosmetic use, for treatment of hair, for treatment of nails, for acute or chronic topical ocular application, for keratoplasty, for laser eye surgery, for induced cornea damage and combinations thereof, the method comprising the step of administering to a subject in need thereof a composition adapted for administration at the site comprising a pharmaceutically effective amount of an N-acylethanolamine.
